(19) United States
(12) Patent Application Publication
(10) Pub. No.: US 2024/0153594 A1
(43) Pub. Date: May 9, 2024

(54) INFORMATION PROCESSING DEVICE AND INFORMATION PROCESSING METHOD

(71) Applicant: Fujitsu Limited, Kawasaki-shi (JP)

(72) Inventor: Satoshi IMAMURA, Kawasaki (JP)

(73) Assignee: Fujitsu Limited, Kawasaki-shi (JP)

(21) Appl. No.: 18/227,996

(22) Filed: Jul. 31, 2023

(30) Foreign Application Priority Data
Nov. 7, 2022 (JP) 2022-178465

(51) Int. Cl. G16C 10/00; G06F 17/11

(52) U.S. Cl. CPC G16C 10/00; G06F 17/11

(57) ABSTRACT

A non-transitory computer-readable recording medium stores a program for causing a computer to execute a process, the process includes determining an execution time for each of one or more algorithms to be used when performing quantum chemical calculation on a target molecule, and determining, based on the determined execution time, first combinations of algorithms that enable the quantum chemical calculation to be performed within a designated time.

### CROSS-REFERENCE TO RELATED APPLICATION

This application is based upon and claims the benefit of priority of the prior Japanese Patent Application No. 2022-178465, filed on Nov. 7, 2022, the entire contents of which are incorporated herein by reference.

### FIELD

The embodiment discussed herein is related to an information processing device and an information processing method.

### BACKGROUND

By calculating the energy of the system from a basis function, which is a function serving as the base of a molecular orbital configuration, and the type of the molecule, it is possible to understand the characteristics of the molecule. Understanding molecular characteristics is useful for drug development, discovery of new materials, and the like. In view of this, quantum chemical calculation for calculating system energy (potential energy) is highly important.

To analyze the characteristics of the system, for example, there is a method for constructing a potential energy surface (PES) by plotting the energy of the target system while changing the molecular structure. Note that, in a case where there is a single parameter, a PES may be referred to as a potential energy curve (PEC). Molecular characteristics may be grasped from a PES or a PEC. Further, there are many algorithms for calculating the energy of the system, and experts select appropriate algorithms in accordance with the target molecule or the like.

Japanese Laid-open Patent Publication No. 2012-032908 is disclosed as related art.

### SUMMARY

According to an aspect of the embodiment, a non-transitory computer-readable recording medium stores a program for causing a computer to execute a process, the process includes determining an execution time for each of one or more algorithms to be used when performing quantum chemical calculation on a target molecule, and determining, based on the determined execution time, first combinations of algorithms that enable the quantum chemical calculation to be performed within a designated time.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an example of quantum chemical calculation;

FIG. 2 is a diagram illustrating an example of various algorithms for energy calculation;

FIG. 3 is a diagram illustrating an example configuration of an information processing device according to an embodiment;

FIG. 4 is a diagram illustrating an example of an evaluation range according to the embodiment;

FIG. 5 is a table illustrating an example of data stored in parameter information according to the embodiment;

FIG. 6 is a diagram illustrating an example results of execution time measurement according to the embodiment;

FIG. 7 is a flowchart illustrating an example flow of a quantum chemical calculation process according to the embodiment; and

FIG. 8 is a diagram illustrating an example hardware configuration of the information processing device according to the embodiment.

### DESCRIPTION OF EMBODIMENT

It is not easy to select an algorithm for calculating the energy of the system. For example, while there is a demand for high-accuracy energy calculation, a realistic time constraint is imposed on the execution time in a trade-off relationship with accuracy, as the scale of the target system becomes larger. Therefore, there are cases where it is difficult to select an executable and highly accurate algorithm.

In the description below, examples of an information processing device and an information processing method according to the embodiment are described with reference to the drawings. Note that the embodiment is not limited by these examples. Furthermore, each of the examples may be appropriately combined within a range without inconsistency.

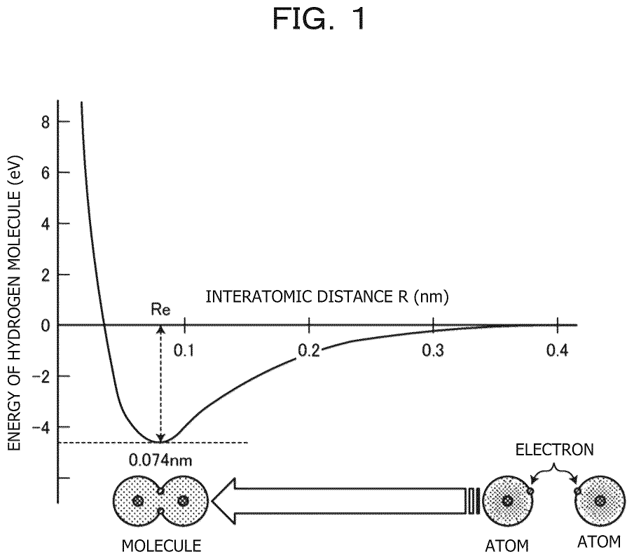

First, quantum chemical calculation as a target of the embodiment is described. FIG. 1 is a diagram illustrating an example of quantum chemical calculation. The graph in FIG. 1 illustrates a potential energy curve (PEC) of a hydrogen molecule, with the horizontal axis indicating interatomic distance, the vertical axis indicating base energy, the target molecule being the hydrogen molecule. As illustrated in FIG. 1, in the quantum chemical calculation, the base energies at a plurality of points are determined by changing the interatomic distance, and thus, a PEC may be obtained by connecting the base energies with a line. As a result, the molecular characteristics of the target molecule may be grasped. For example, as illustrated in FIG. 1, the maximal point of the PEC indicates a transition state in which atoms are separated from each other. On the other hand, the minimal point of the PEC indicates a stable state in which atoms are bonded to each other, and the target molecule is generated. In this manner, it is possible to understand the characteristics of the target molecule by calculating the energy of the target molecule. Note that, although the PEC has been described as an example with reference to FIG. 1, the characteristics of the target molecule may also be grasped by obtaining the energy of the target molecule with a potential energy surface (PES).

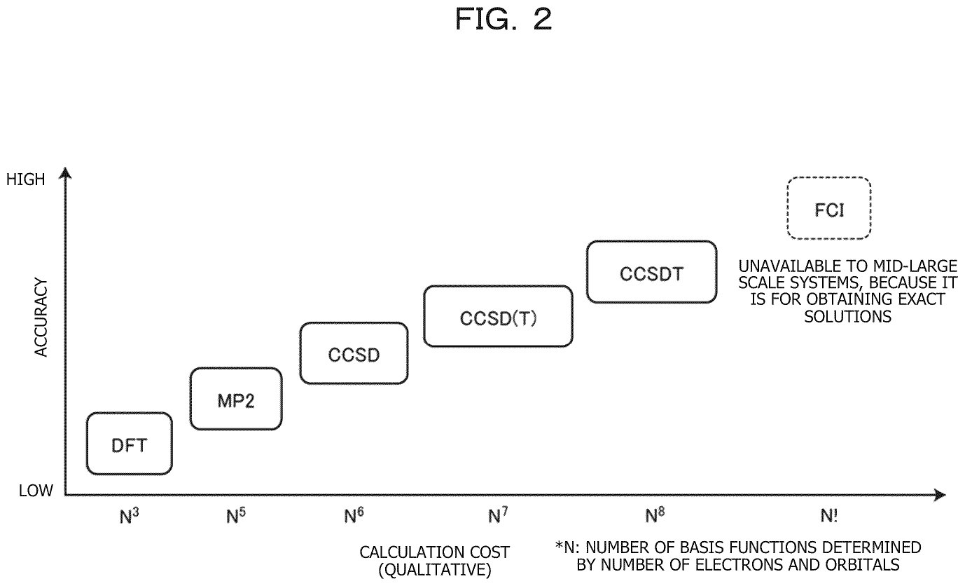

However, there are many algorithms for calculating the energy of a target molecule. FIG. 2 is a diagram illustrating an example of various algorithms for energy calculation. As illustrated in FIG. 2, examples of algorithms for calculating the energy of a target molecule include the density functional theory method (DFT), the Moller-Plesset perturbation method to second order (MP2), the coupled-cluster method with single and double excitations (CCSD), and the like. Although the energy of a target molecule may be calculated by the algorithms illustrated in FIG. 2, calculation costs and accuracies vary with the algorithms as illustrated in FIG. 2. Furthermore, the calculation cost and the accuracy of energy calculation are in a trade-off relationship. Therefore, in the embodiment, an algorithm that may be executed with a realistic time constraint and has a higher accuracy is selected.

### Functional Configuration of an Information Processing Device

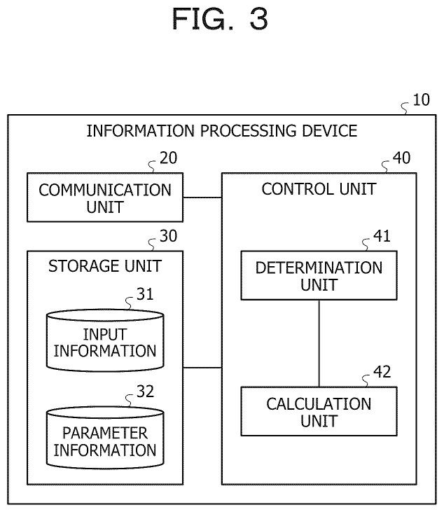

Next, the functional configuration of an information processing device that executes an information processing method of the embodiment is described. FIG. 3 is a diagram illustrating an example configuration of an information processing device 10 according to the embodiment. The information processing device 10 illustrated in FIG. 3 is a server computer, or an information processing device such as a desktop personal computer (PC) or a notebook PC, for example. Note that FIG. 3 illustrates the information processing device 10 as one computer. However, the information processing device 10 may be a distributed computing system including a plurality of computers. Alternatively, the information processing device 10 may be a cloud computer device being managed by a service provider that provides cloud computing services.

As illustrated in FIG. 3, the information processing device 10 includes a communication unit 20, a storage unit 30, and a control unit 40.

The communication unit 20 is a processing unit that controls communication with another device, and is a communication interface such as a network interface card, or a universal serial bus (USB) interface, for example.

The storage unit 30 has a function of storing various kinds of data and a program to be executed by the control unit 40, and is implemented with a storage device such as a memory or a hard disk, for example. The storage unit 30 stores input information 31, parameter information 32, and the like.

In the input information 31, input information that is input at a time of quantum chemical calculation or the like is stored, for example. The input information may be, for example, molecular information including a basis function that is a function serving as the base of a molecular orbital configuration and the type of the molecule, and information regarding an evaluation range, a time constraint, and the like in the quantum chemical calculation.

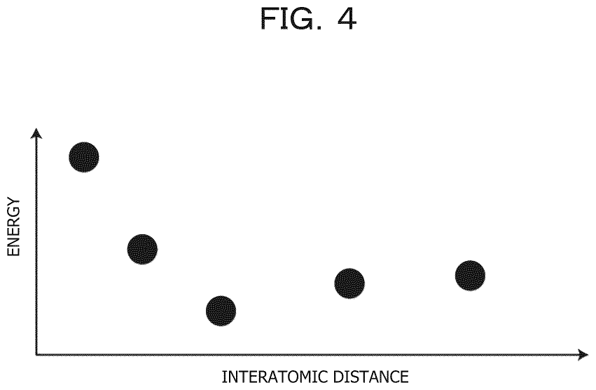

Here, the evaluation range stored in the input information 31 may be, for example, 20 points at intervals of 0.1 Å (angstrom) between interatomic distances of 0.5 to 2.4 Å on a PEC as illustrated in FIG. 1, or the like. FIG. 4 is a diagram illustrating an example of the evaluation range according to the embodiment. FIG. 4 illustrates an example in which five points between predetermined interatomic distances on a PEC are designated as the evaluation range by the user.

Furthermore, the time constraint stored in the input information 31 may be, for example, 400 or 100 seconds (sec) designated by the user. Note that the input information such as the evaluation range and the time constraint designated by the user may be input via, for example, an information processing terminal being used by the user. In the embodiment, a more accurate quantum chemical calculation algorithm that may be executed with a time constraint for each of the points designated as the evaluation range by the user is selected. Accordingly, the time constraint designated by the user is a constraint on the total execution time of the algorithm at each of the points designated as the evaluation range by the user. Note that the execution time indicates, for example, the time to be taken for the algorithm to obtain the potential energy at the point of a certain interatomic distance.

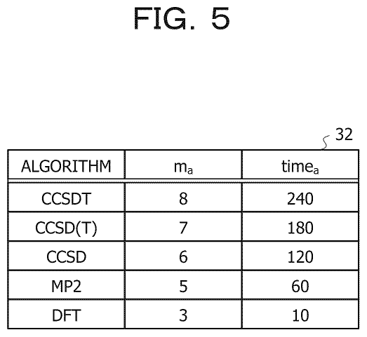

In the parameter information 32, for example, a regression coefficient, an execution time, and the like, which are accuracy indexes of the respective algorithms to be used for quantum chemical calculation, are stored. FIG. 5 is a table illustrating an example of data stored in the parameter information 32 according to the embodiment. As illustrated in FIG. 5, for example, “algorithm”, “mₐ”, and “timeₐ” are associated with one another and are stored in the parameter information 32. An “algorithm” may be, for example, an identifier, a name, or the like for uniquely identifying a predetermined algorithm a to be used for quantum chemical calculation. An “mₐ” is, for example, a value corresponding to accuracy, for example, a regression coefficient that is an index of accuracy and may be a value indicating the calculation cost of an algorithm a. A “timeₐ” may be, for example, a value indicating the execution time predicted for the algorithm a.

Note that the above information stored in the storage unit 30 is merely an example, and the storage unit 30 may store various kinds of information other than the above information.

The control unit 40 is a processing unit that is in charge of overall control of the information processing device 10, and is a processor or the like, for example. The control unit 40 includes a determination unit 41 and a calculation unit 42. Note that each of the processing units is an example of an electronic circuit included in a processor, or an example of a process to be performed by the processor.

For example, when quantum chemical calculation is performed on the target molecule with one or more algorithms, the determination unit 41 determines an execution time, and a value corresponding to accuracy for each algorithm to be used. To determine the execution time of each algorithm, the determination unit 41 uses, for example, a single regression model that predicts the execution time indicated by the following Expression (1).

log₁₀(timeₐ)=mₐ·Log₁₀(N)+cₐ (1)

In Expression (1), timeₐ represents the execution time predicted for the algorithmₐ. Meanwhile, N represents the number of basis functions determined by the number of electrons and the orbitals, and may be acquired from the molecular information and the basis functions. Further, mₐ represents the regression coefficient, and is the index of the calculation cost of the algorithm a. Furthermore, cₐ represents a constant term. Methods for determining the regression coefficient mₐ and the constant term cₐ are now described.

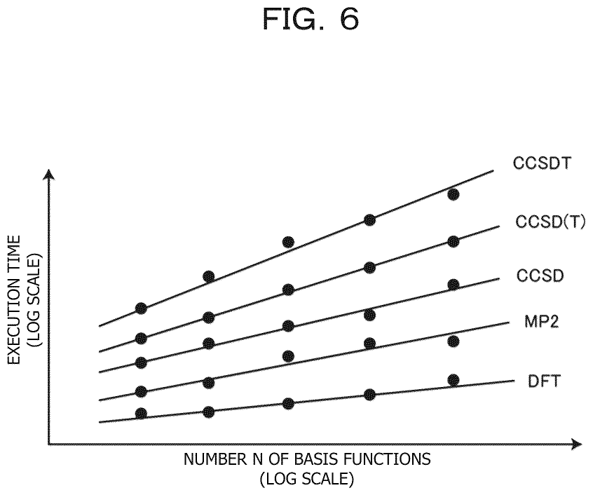

First, the determination unit 41 measures the execution time of each of the algorithms such as the algorithm a, by changing the number N of basis functions of the molecule serving as the benchmark, for example. To change the number N of basis functions, the basis functions are changed, for example. Referring now to FIG. 6, an example of results of execution time measurement is described.

FIG. 6 is a diagram illustrating an example of results of execution time measurement according to the embodiment. Graphs in FIG. 6 are graphs illustrating the execution time of respective algorithms on a log scale, the execution time being measured by changing the number N of basis functions of the molecule serving as the benchmark. Also, the horizontal axis indicates the number N of basis functions on a log scale. The number N of basis functions increases with the scale of the basis functions.

Then, the determination unit 41 determines the regression coefficient mₐ and the constant term cₐ of the algorithm a, using single regression analysis from the measurement results illustrated in FIG. 6 or the like, for example. In the example in FIG. 6, the regression coefficient mₐ and the constant term cₐ are determined for each of five types of algorithms.

In this manner, the determination unit 41 determines each parameter of the single regression model as indicated by Expression (1) for each algorithm to be used as a possible choice, and constructs a single regression model. Then, the determination unit 41 predicts and determines the execution time of quantum chemical calculation for the target molecule, using the constructed single regression model, based on the determined regression coefficient mₐ and constant term cₐ for each algorithm to be used as a possible choice. Note that the determined regression coefficient and the predicted execution time may be respectively stored in “mₐ” and “timeₐ” in the parameter information 32 for each algorithm.

Also, the determination unit 41 determines a combination of algorithms with which quantum chemical calculation may be performed within a designated time, based on the determined regression coefficient mₐ and the execution time determined by prediction, for example. This includes determining a combination of algorithms, for example, for each point designated as an evaluation range on a PES or PEC, by determining an algorithm with which quantum chemical calculation may be performed within a designated time, or with a designated time constraint, for example. Furthermore, the determined regression coefficient mₐ and the execution time determined by prediction are values respectively stored in “mₐ” and “timeₐ” for each algorithm as illustrated in FIG. 5, for example.

For example, the determination unit 41 determines a combination of algorithms by solving a linear programming problem expressed as below by Expression (2), for example, and having maximization of accuracy as an objective function, for the points designated as the evaluation range. Note that the points designated as the evaluation range are the five points illustrated in FIG. 4, for example.

$$Objective function : max {\sum }_{p ∈ P} {\sum }_{a ∈ A} {m}_{a , p} \cdot {x}_{a , p} \qquad ( 2 )$$

In Expression (2), p represents the point p included in a set P of points on a PES or PEC, the set being designated as the evaluation range. Also, a represents the algorithm a included in a set A of quantum chemical calculation algorithms. Further, m_{a,p} represents the regression coefficient of the algorithm a for calculating the potential energy of the point p, and indicates the accuracy of the algorithm a. As illustrated in FIG. 2, the higher the calculation cost N^{ma}, the higher the accuracy. Accordingly, the regression coefficient mₐ of each algorithm is regarded as an accuracy index, for example. The regression coefficient mₐ of the algorithm a is determined for each algorithm, for example, and is stored beforehand in the parameter information 32. Furthermore, x_{a,p} indicates 1 in a case where the potential energy of the point p is obtained by the algorithm a, and indicates 0 in other cases as expressed by the following Expression (3).

$${x}_{a , p} = \{ \begin{matrix}1 , & if algorithm a evaluates point p \\ 0 , & otherwise\end{matrix} \qquad ( 3 )$$

Further, the algorithm for calculating the potential energy of the point p is determined to be one algorithm included in the set A of quantum chemical calculation algorithms. Accordingly, a constraint expressed by the following Expression (4) is imposed on x_{a,p}.

$$Constraint : {\sum }_{a ∈ A} {x}_{a , p} = 1 \vert p ∈ P \qquad ( 4 )$$

Furthermore, the time within the time designated by the user, that is, the time constraint is expressed by the following Expression (5).

$${time}_{total} = {\sum }_{p ∈ P} {\sum }_{a ∈ A} {time}_{a , p} \cdot {x}_{a , p} \leq {time}_{limit} \qquad ( 5 )$$

In Expression (5), time_{a,p} represents the execution time predicted for the algorithm a for calculating the potential energy of the point p. The execution time timeₐ predicted for the algorithm a is predicted and determined for each algorithm, for example, and is stored beforehand in the parameter information 32. Further, timeₗᵢₘᵢₜ is the time constraint designated by the user. For example, the determination unit 41 uses Expression (5), to perform control so that the total time timeₜₒₜₐₗ of obtaining the potential energy of all points in the evaluation range designated by the user falls within the time constraint timeₗᵢₘᵢₜ.

Furthermore, the determination unit 41 may use the following Expression (6), to perform control so that the potential energy is obtained with higher accuracy when, for example, the value of the point is greater, the interatomic distance being long, the interatomic angle (also referred to as “bond angle”) being large, or the like.

m_{a,i}≤m_{a,j}|a∈A,i<j∈P (6)

In Expression (6), i and j represent the respective points included in the set P of points on the PES or PEC, and j represents a point having a greater value, such as a longer interatomic distance or a larger bond angle, than i.

The determination unit 41 uses Expressions (2) to (6), to determine first combinations of algorithms with which quantum chemical calculation may be performed with the designated time constraint. Also, the determination unit 41 determines the combination having the highest value corresponding to accuracy among the determined first combinations.

Following Expressions (7) and (8) are expressions that indicate the value of the objective function and the total execution time, respectively, in a case where a combination of algorithms for calculating the potential energy of the five points as illustrated in FIG. 4 is determined based on the parameter information as illustrated in FIG. 5, the time constraint being 400 seconds.

$${\sum }_{p ∈ P} {\sum }_{a ∈ A} {m}_{a , p} \cdot {x}_{a , p} = 24 \qquad ( 7 )$$

$${\sum }_{p ∈ P} {\sum }_{a ∈ A} {time}_{a , p} \cdot {x}_{a , p} = 380 \qquad ( 8 )$$

In Expressions (7) and (8), values stored in “mₐ” and “timeₐ” in the parameter information 32 may be used as the accuracy m_{a,p} and the execution time time_{a,p} of the algorithm a, respectively. An algorithm for calculating the potential energy of each point is determined so that the accuracy is maximized with the objective function of Expression (7), and the total execution time of Expression (8) becomes equal to or shorter than 400 seconds. As a result, [DFT, DFT, MP2, CCSD, CCSD(T)] is determined as the combination of algorithms, for example. Note that the combination of algorithms [DFT, DFT, MP2, CCSD, CCSD(T)] is used as an algorithm for calculating the potential energy of each of the points, starting from the left one of the five points as illustrated in FIG. 4, for example. Further, the solutions of Expressions (7) and (8) are the sums of the values stored in “mₐ” and “timeₐ”, respectively, in the parameter information 32 corresponding to each determined algorithm.

As another example, following Expressions (9) and (10) are expressions that indicate the value of the objective function and the total execution time, respectively, in a case where a combination of algorithms for calculating the potential energy of the five points is determined based on the parameter information as illustrated in FIG. 5, the time constraint being 100 seconds.

$${\sum }_{p ∈ P} {\sum }_{a ∈ A} {m}_{a , p} \cdot {x}_{a , p} = 17 \qquad ( 9 )$$

$${\sum }_{p ∈ P} {\sum }_{a ∈ A} {time}_{a , p} \cdot {x}_{a , p} = 100 \qquad ( 10 )$$

With Expressions (9) and (10), [DFT, DFT, DFT, DFT, MP2] is determined as a combination of algorithms, for example. Note that, in the examples described with reference to Expressions (7) to (10), a combination of algorithms is formed with algorithms of a plurality of types. However, there might be cases where an entire combination of algorithms is formed with identical algorithms.

### Flow of Processing

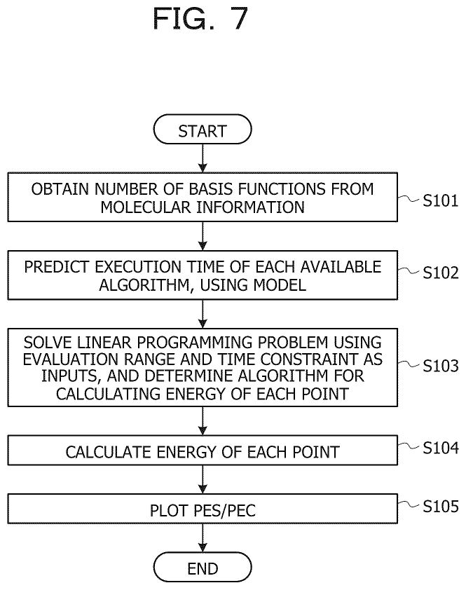

Next, a quantum chemical calculation process to be performed by the information processing device 10 is described with reference to FIG. 7. FIG. 7 is a flowchart illustrating an example flow of a quantum chemical calculation process according to the embodiment. The quantum chemical calculation process illustrated in FIG. 7 is started, for example, in response to a user's input of information such as molecular information including basis functions and the type of the molecule, the evaluation range, and the time constraint as input data to the information processing device 10. Also, as described above with reference to FIG. 6 and others, it is assumed that the regression coefficient mₐ and the constant term cₐ of the single regression model of each algorithm available in the quantum chemical calculation have been determined through a single regression analysis, and a single regression model has been constructed in advance.

First, as illustrated in FIG. 7, the information processing device 10 obtains the number of basis functions, in accordance with the basis functions and the type of molecule included in molecular information input as input data, for example (step S101).

Next, the information processing device 10 predicts and determines the execution time of each algorithm available in quantum chemical calculation, for example, using the single regression model constructed in advance (step S102).

Next, the information processing device 10 solves a linear programming problem using an evaluation range and a time constraint as inputs, for example, and determines an algorithm for calculating the potential energy of each point on a PES or PEC (step S103). The linear programming problem is, for example, a linear programming problem that is expressed by Expression (2) and has maximization of accuracy as an objective function.

Next, using the algorithm determined in step S103, for example, the information processing device 10 calculates the potential energy of each point on the PES or PEC (step S104).

Next, the information processing device 10 plots the PES or PEC, based on the potential energy calculated in step S104, for example (step S105). After the execution of step S105, the quantum chemical calculation process illustrated in FIG. 7 ends.

### Effects

As described above, when performing quantum chemical calculation on the target molecule with one or more algorithms, the information processing device 10 determines an execution time for each algorithm to be used, and determines first combinations of algorithms with which the quantum chemical calculation may be performed within a designated time, based on the determined execution times.

In this manner, the information processing device 10 determines an execution time for each algorithm, and determines a combination of algorithms based on the determined execution times. Thus, the information processing device 10 may determine a quantum chemical calculation algorithm that may be executed within a prescribed time.

Further, the information processing device 10 determines a value corresponding to accuracy for each algorithm, and determines the combination having the highest value corresponding to accuracy among the first combinations.

Thus, the information processing device 10 may determine a high-accuracy quantum chemical calculation algorithm that may be executed within a prescribed time.

Furthermore, based on the determined first combinations, the information processing device 10 performs quantum chemical calculation on the target molecule, to calculate the potential energy of the target molecule.

As a result, the information processing device 10 may calculate potential energy within a prescribed time.

Meanwhile, the process to be performed by the information processing device 10 to determine an execution time includes a process of: measuring an execution time by changing the number of basis functions of the molecule serving as the benchmark, for each algorithm to be used; determining a regression coefficient and a constant term that are to serve as accuracy indexes, using a single regression analysis for each algorithm to be used; and predicting and determining the execution time of quantum chemical calculation for the target molecule, based on the determined regression coefficient and constant term, for each algorithm to be used.

Thus, the information processing device 10 may determine a high-accuracy quantum chemical calculation algorithm that may be executed within a prescribed time.

Further, the process to be performed by the information processing device 10 to determine the first combinations includes a process of determining the first combinations by determining, based on the determined execution time, an algorithm that enables quantum chemical calculation to be performed within a designated time, for each designated point on a potential energy surface or a potential energy curve.

Thus, the information processing device 10 may determine a higher-accuracy quantum chemical calculation algorithm that may be executed within a prescribed time.

### System

Pieces of information including the processing procedures, the control procedures, the specific names, the various kinds of data, and the parameters described above or illustrated in the drawings may be changed as appropriate, unless otherwise specified. Further, the specific examples, distributions, numerical values, and the like described in the embodiment are merely examples, and may be changed as appropriate.

Furthermore, specific forms of dispersion and integration of the components of the information processing device 10 are not limited to those illustrated in the drawings. For example, the determination unit 41 of the information processing device 10 may be dispersed in a plurality of processing units, or the determination unit 41 and the calculation unit 42 of the information processing device 10 may be integrated into one processing unit. For example, all or some of the components may be functionally or physically dispersed or integrated in optional units, depending on various kinds of loads, use situations, or the like. Moreover, all or some of the respective processing functions of each device may be implemented by a central processing unit (CPU) and a program to be analyzed and executed by the CPU, or may be implemented as hardware by wired logic.

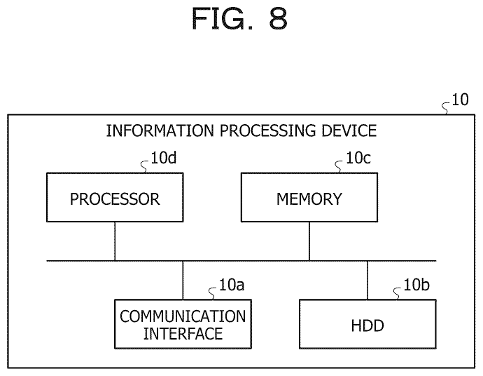

FIG. 8 is a diagram illustrating an example hardware configuration of the information processing device 10 according to the embodiment. As illustrated in FIG. 8, the information processing device 10 includes a communication interface 10a, a hard disk drive (HDD) 10b, a memory 10c, and a processor 10d. Further, the respective components illustrated in FIG. 8 are coupled to each other by a bus or the like.

The communication interface 10a is a network interface card or the like, and communicates with another information processing device. The HDD 10b stores a program and data for operating the respective functions of the information processing device 10, for example.

The processor 10d is a CPU, a micro processing unit (MPU), a graphics processing unit (GPU), or the like. Furthermore, the processor 10d may be implemented by an integrated circuit such as an application specific integrated circuit (ASIC) or a field programmable gate array (FPGA). The processor 10d reads, from the HDD 10b or the like, a program for performing processes similar to those of the respective processing units illustrated in FIG. 3 and others, and loads the read program into the memory 10c, for example. By doing so, the processor 10d may operate as a hardware circuit that performs a process of implementing the respective functions of the information processing device 10.

Also, the information processing device 10 may implement functions similar to the functions of the above embodiment by reading the above program from a recording medium with a medium reading device and executing the above read program. Note that the program is not limited to a program to be executed by the information processing device 10. For example, the above embodiment may also be applied in a case where another information processing device executes the program, or a case where another information processing device and the information processing device 10 cooperate to execute the program.

The program may be distributed via a network such as the Internet. Alternatively, the program may be stored in a computer-readable recording medium such as a hard disk, a flexible disk (FD), a compact disc read only memory (CD-ROM), a magneto-optical disk (MO), or a digital versatile disc (DVD). Then, the program may be read from the recording medium and be executed by the information processing device 10 or the like.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a program for causing a computer to execute a process, the process comprising:
determining an execution time for each of one or more algorithms to be used when performing quantum chemical calculation on a target molecule; and
determining, based on the determined execution time, first combinations of algorithms that enable the quantum chemical calculation to be performed within a designated time.

2. The non-transitory computer-readable recording medium according to claim 1, the process further comprising:
determining a value corresponding to accuracy, for each of the one or more algorithms; and
determining a combination having a highest value corresponding to accuracy among the first combinations.

3. The non-transitory computer-readable recording medium according to claim 1, the process further comprising:
calculating a potential energy of the target molecule by performing the quantum chemical calculation on the target molecule, based on the determined first combinations.

4. The non-transitory computer-readable recording medium according to claim 2, the process further comprising:
measuring the execution time by changing a counted number of basis functions of a molecule that serve as benchmarks, for each of the one or more algorithms;
determining a regression coefficient and a constant term that serve as accuracy indexes using a single regression analysis, for each of the one or more algorithms; and
predicting and determining the execution time of the quantum chemical calculation for the target molecule, based on the determined regression coefficient and the determined constant term, for each of the one or more algorithms.

5. The non-transitory computer-readable recording medium according to claim 1, the process further comprising:
determining the first combinations by determining, based on the determined execution time, algorithms that enable the quantum chemical calculation to be performed within the designated time, for each designated point on a potential energy surface or a potential energy curve.

6. An information processing device, comprising:
a memory; and
a processor coupled to the memory and the processor configured to:
determine an execution time for each of one or more algorithms to be used when performing quantum chemical calculation on a target molecule; and
determine, based on the determined execution time, first combinations of algorithms that enable the quantum chemical calculation to be performed within a designated time.

7. The information processing device according to claim 6, wherein
the processor is further configured to:
determine a value corresponding to accuracy, for each of the one or more algorithms; and
determine a combination having a highest value corresponding to accuracy among the first combinations.

8. The information processing device according to claim 6, wherein
the processor is further configured to:
calculate a potential energy of the target molecule by performing the quantum chemical calculation on the target molecule, based on the determined first combinations.

9. The information processing device according to claim 7, wherein
the processor is further configured to:
measure the execution time by changing a counted number of basis functions of a molecule that serve as benchmarks, for each of the one or more algorithms;
determine a regression coefficient and a constant term that serve as accuracy indexes using a single regression analysis, for each of the one or more algorithms; and
predict and determine the execution time of the quantum chemical calculation for the target molecule, based on the determined regression coefficient and the determined constant term, for each of the one or more algorithms.

10. The information processing device according to claim 6, wherein
the processor is further configured to:
determine the first combinations by determining, based on the determined execution time, algorithms that enable the quantum chemical calculation to be performed within the designated time, for each designated point on a potential energy surface or a potential energy curve.

11. An information processing method, comprising:
determining, by a computer, an execution time for each of one or more algorithms to be used when performing quantum chemical calculation on a target molecule; and
determining, based on the determined execution time, first combinations of algorithms that enable the quantum chemical calculation to be performed within a designated time.

12. The information processing method according to claim 11, the process further comprising:
determining a value corresponding to accuracy, for each of the one or more algorithms; and
determining a combination having a highest value corresponding to accuracy among the first combinations.

13. The information processing method according to claim 11, the process further comprising:
calculating a potential energy of the target molecule by performing the quantum chemical calculation on the target molecule, based on the determined first combinations.

14. The information processing method according to claim 12, the process further comprising:
measuring the execution time by changing a counted number of basis functions of a molecule that serve as benchmarks, for each of the one or more algorithms;
determining a regression coefficient and a constant term that serve as accuracy indexes using a single regression analysis, for each of the one or more algorithms; and
predicting and determining the execution time of the quantum chemical calculation for the target molecule, based on the determined regression coefficient and the determined constant term, for each of the one or more algorithms.

15. The information processing method according to claim 11, the process further comprising:
determining the first combinations by determining, based on the determined execution time, algorithms that enable the quantum chemical calculation to be performed within the designated time, for each designated point on a potential energy surface or a potential energy curve.
